(19) United States
(12) Patent Application Publication
(10) Pub. No.: US 2008/0051433 A1
(43) Pub. Date: Feb. 28, 2008

(54) Pyrrole-3-Carboxamide Derivatives for the Treatment of Obesity

(76) Inventors: Magnus Bjorsne, Molndal (SE); Leifeng Cheng, Molndal (SE); Thomas Elebring, Molndal (SE); Anna Maria Boije, Molndal (SE); Eva-Lotte Lindstedt-Alstermark, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 10/588,518

(22) PCT Filed: Feb. 17, 2005
(86) PCT No.: PCT/GB05/00588

§ 371 Date: May 29, 2007

(30) Foreign Application Priority Data
Feb. 20, 2004 (GB) 0403780.0

(51) Int. Cl. A61K 31/445; A61P 25/00; A61P 3/04; C07D 401/00

(52) U.S. Cl. 514/326; 546/208

(57) ABSTRACT

The present invention relates to compounds of Formula (I) and processes for preparing such compounds, their use in the treatment of obesity, psychiatric and neurological disorders, to methods for their therapeutic use and to pharmaceutical compositions containing them.

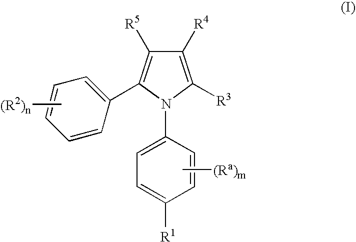

### FIELD OF INVENTION

The present invention relates to certain compounds of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WOO1/70700 and EP 656354).

WO 03/027069 discloses pyrrole -3-carboxamides of formula A

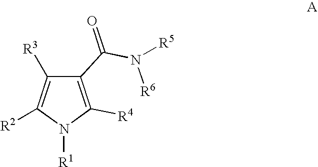

in which R¹ and R² are each a phenyl group, optionally substituted with one or more is halogen, (C₁-C₆)alkyl, (C₁-C₆)alkoxy , trifluoromethyl, hydroxy, cyano or nitro; R³ is hydrogen; R⁴ is CH₃; R⁵ is hydrogen or (C₁-C₆)alkyl; R⁶ is cyclohexyl, (C₁-C₆)alkyl, cyclopentyl, cycloheptyl or cyclo(C₃-C₇)alkyl(C₁-C₃)alkyl, benzyl, phenyl, piperidin-4-yl, piperidin-3-yl, or pyrrolidin-3-yl each of which is optionally substituted or a group NR⁷R⁸ where R⁷ is hydrogen or (C₁-C₆)alkyl; and R⁸ is (C₁-C₆)alkyl or phenyl each of which is optionally substituted or R⁷ and R⁸ taken together with the nitrogen to which they are attached form a 5- to 10-membered saturated heterocyclic radical which is optionally substituted; or R⁵ and R⁶ taken together with the nitrogen to which they are attached form a 5- to 10- membered saturated heterocyclic radical which is optionally substituted wherein the optional substituents include (C₁-C₆)alkyl and (C₁-C₆)alkoxy; are C₁ modulators.

Co-pending application PCT/GB03/05569 (WO2004/058249) discloses pyrrole-3-carboxamides of formula B

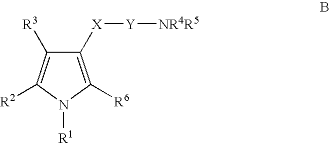

and pharmaceutically acceptable salts, prodrugs, solvates and crystalline forms thereof, in which

R¹ and R² independently represent phenyl, thienyl or pyridyl each of which is optionally substituted by one, two or three groups represented by Z;

Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, mono or di C₁₋₃alkylamido, C₁₋₃alkylsulphonyl, C₁₋₃alkoxycarbonyl, carboxy, cyano, carbamoyl, mono or di C₁₋₃alkyl carbamoyl, sulphamoyl and acetyl; and

R³ is H, a C₁₋₃alkyl group, a C₁₋₃alkoxymethyl group, trifluoromethyl, a hydroxyC₁₋₃alkyl group, an aminoC₁₋₃alkyl group, C₁₋₃alkoxycarbonyl, carboxy, cyano, carbamoyl, mono or di C₁₋₃alkylcarbamoyl, acetyl, or hydrazinocarbonyl of formula —CONHNR^{a}R^{b} wherein R^{a} and R^{b} are as defined for R⁴ and R⁵ respectively and;

X is CO or SO₂;

Y is absent or represents NH optionally substituted by a C₁₋₃alkyl group;

R⁴ and R⁵ independently represent:

a C₁₋₆alkyl group;

an (amino)C₁₋₄alkyl-group in which the amino is optionally substituted by one or more C₁₋₃alkyl groups;

an optionally substituted non-aromatic C₃₋₁₅carbocyclic group;

a (C₃₋₁₂cycloalkyl)C₁₋₃alkyl-group;

a group —CH₂)ᵣ(phenyl)ₛ in which r is 0, 1, 2, 3 or 4, s is 1 when r is 0 otherwise s is 1 or 2 and the phenyl groups are optionally independently substituted by one, two or three groups represented by Z;

naphthyl;

anthracenyl;

a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;

1-adamantylmethyl;

a group—(CH₂)ₜ Het in which t is 0, 1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo;

or R⁴ represents H and R⁵ is as defined above;

or R⁴ and R⁵ together with the nitrogen atom to which they are attached represent a saturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy or benzyl;

R⁶ is H, a C₁₋₃alkyl group, a C₁₋₃alkoxymethyl group, trifluoromethyl, a hydroxyC₁₋₃alkyl group, an aminoC₁₋₃alkyl group, C₁₋₃alkoxycarbonyl, carboxy, cyano, carbamoyl, mono or di C₁₋₃alkylcarbamoyl, acetyl, or hydrazinocarbonyl of formula —CONHNR^{a}R^{b} wherein R^{a} and R^{b} are as defined for R⁴ and R⁵ respectively and;

with the proviso that when R⁶ is methyl then the group X—Y-NR⁴R⁵ does not represent CONHC₆H₁₃, CONHC₁₂H₂₅, CONH₂, CONHCH₃ , CON(CH₃)₂,

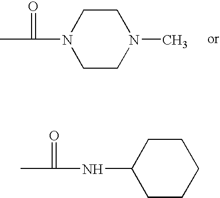

and with the further proviso that when R¹ and R² independently represent phenyl then Z is not an ortho methyl group as CB₁ modulators. Nanoparticles of some of these compounds are disclosed in WO2004/069227.

Co-pending application WO2004/060870 discloses pyrrole-3-carboxamides as CB1 receptor inverse agonists. The compounds exemplified are mostly 1-cycloalkylmethylene derivatives or 1 -benzyl derivatives.

However, there is a need for CB₁ modulators with improved potency, selectivity, physicochemical properties and/or DMPK properties and/or pharmacodynamic properties.

### DESCRIPTION OF THE INVENTION

The invention relates to a compound of formula (I)

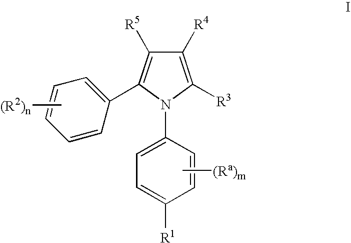

and pharmaceutically acceptable salts and solvates thereof, in which R¹ represents a) a C₃₋₆alkoxy group substituted by one or more fluoro, b) a group of formula phenyl(CH₂)ₚO— in which p is 1, 2 or 3 and the phenyl ring is optionally substituted by 1, 2 or 3 groups represented by Z, c) a group R⁶S(O)₂O or R⁶S(O)₂NH in which R⁶ represents a C₁₋₆alkyl group optionally substituted by one or more fluoro, or R⁶ represents phenyl or a heteroaryl group each of which is optionally substituted by 1, 2 or 3 groups represented by Z or d) a group of formula (R⁷)₃ Si in which R⁷ represents a C₁₋₆alkyl group which may be the same or different;

R^{a} represents halo, a C₁₋₃alkyl group or a C₁₋₃alkoxy group

m is 0, 1, 2or3;

R² represents a C₁₋₃alkyl group, a C₁₋₃alkoxy group, hydroxy, nitro, cyano or halo n is 0, 1, 2 or 3;

R³ represents H, a C₁₋₆alkyl group, a C₁₋₆alkoxy group or a C₁₋₆alkoxyC₁₋₆alkylene group which contains a maximum of 6 carbon atoms, each of which groups is optionally substituted by one or more fluoro or cyano;

R⁴ represents

a) a group X—Y-NR⁸R⁹

in which X is CO or SO₂,

Y is absent or represents NH optionally substituted by a C₁₋₃alkyl group; and R⁸ and R⁹ independently represent:

a C₁₋₆alkyl group optionally substituted by 1, 2, or 3 groups represented by W;

a C₃₋₁₅cycloalkyl group optionally substituted by 1, 2, or 3 groups represented by W;

an optionally substituted (C₃₋₁₅cycloalkyl)C₁₋₃alkylene group optionally substituted by 1, 2, or 3 groups represented by W;

a group —CH₂)ᵣ(phenyl )₂ in which r is 0, 1, 2, 3 or 4, s is 1 when r is 0 otherwise s is 1 or 2 and the phenyl groups are optionally independently substituted by one, two or three groups represented by Z;

a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;

a group —(CH₂)ₜ Het in which t is 0, 1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo;

or R⁸ represents H and R⁹ is as defined above;

or R⁸ and R⁹ together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro or benzyl;

or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;

R⁵ represents H or a C₁₋₃alkyl group;

Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and

W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperdinyl or piperazinyl in which the heterocyclic amine is optionally substituted by a C₁₋₃alkyl group or hydroxyl.

It will be understood that where a substituent Z is present in more than one group that these substituents are independently selected and may be the same or different. The same is true for W. Similarly when m 2 or 3 then the groups R^{a} are independently selected so that they may be the same or different and similarly when n is 2 or 3 then the groups R² are independently selected so that they may be the same or different.

The term C₃₋₁₅cycloalkyl includes monocyclic, bicyclic, tricyclic and spiro systems for example, cyclopentyl, cyclohexyl and adamantyl.

The term heteroaryl means an aromatic 5-, 6-, or 7-membered monocyclic ring or a 9- or 10-membered bicyclic ring, with up to five ring heteroatoms selected from oxygen, nitrogen and sulfur. Suitable aromatic heteroaryl groups include, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl. Preferably furyl, pyrrolyl, thienyl, oxazolyl, isoxazolyl, imidazolyl, pyrazolyl, oxazolyl thiazolyl, isothiazolyl, oxadiazolyl, thiadiazolyl, triazolyl, tetrazolyl, pyridyl, pyridazinyl, pyrimidinyl, pyrazinyl or 1,3,5-triazenyl and more preferably pyrrolyl, thienyl, imidazolyl, oxazolyl or pyridyl.

Suitable saturated or partially unsaturated 5 to 8 membered heterocyclic groups containing one or more heteroatoms selected from nitrogen, oxygen or sulphur include, for example tetrahydrofuranyl, tetrahydropyranyl, 2,3-dihydro-1,3-thiazolyl, 1,3-thiazolidinyl, pyrrolinyl, pyrrolidinyl, morpholinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl or tetrahydropyrimidinyl, preferably tetrahydrofuranyl, tetrahydropyranyl, pyrrolidinyl, morpholinyl, piperidinyl or piperazinyl, more preferably tetrahydrofuran-3-yl, tetrahydropyran-4-yl, pyrrolidin-3-yl, morpholino, piperidino, piperidin-4-yl or piperazin-1-yl.

A particular group of compounds of formula I is represented by formula IA

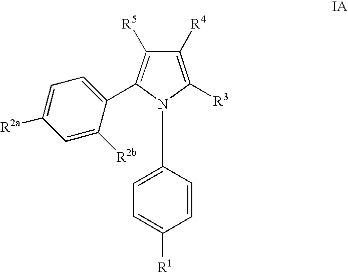

in which R¹ is

a) a C₃₋₆alkoxy group substituted by one or more fluoro, b) a group of formula phenyl(CH₂)ₚO— in which p is 1, 2 or 3 and the phenyl ring is optionally substituted by 1, 2 or 3 groups represented by Z, c) a group R⁶S(O)₂O or R⁶S(O)₂NH in which R⁶ represents a C₁₋₆alkyl group optionally substituted by one or more fluoro, or R⁶ represents phenyl or a heteroaryl group each of which is optionally substituted by 1, 2 or 3 groups represented by Z or d) a group of formula (R⁷)₃ Si in which R⁷ represents a C₁₋₆alkyl group which may be the same or different;

R^{2a} represents chloro;

R^{2b} represents chloro;

R³ represents a C₁₋₃alkyl group;

R⁴ represents a group CONHNR⁸R⁹ in which NR⁸R⁹ represents piperidino; and

R⁵ represents H.

In one particular group of compounds of formula I or formula IA, R¹ represents a C₃₋₆alkoxy group substituted by one or more fluoro.

In another particular group of compounds of formula I or formula IA, R¹ represents a group of formula phenyl(CH₂)ₚO— in which p is 1, 2 or 3 and the phenyl ring is optionally substituted by 1, 2 or 3 groups represented by Z.

In a further particular group of compounds of formula I or formula IA, R¹ represents a group R⁶S(O)₂O or R⁶S(O)₂NH in which R⁶ represents a C₁₋₆alkyl group optionally substituted by one or more fluoro, or R⁶ represents phenyl or a heteroaryl group each of which is optionally substituted by 1, 2 or 3 groups represented by Z.

In a still further particular group of compounds of formula I or formula IA, R¹ represents a group of formula (R⁷)₃ Si in which R⁷ represents a C₁₋₆alkyl group which may be the same or different.

Further values of R¹ in compounds of formula I and formula IA now follow. It will be understood that such values may be used where appropriate with any of the definitions, claims or embodiments defined hereinbefore or hereinafter.

Particularly R¹ is a group R⁶S(O)₂O in which R⁶ represents a C₁₋₆alkyl group optionally substituted by one or more fluoro. More particularly R¹ is benzyloxy, trifluoromethylsulphonyloxy, 3,3,3-trifluoropropoxy, n-butylsulfonyloxy, n-propylsulfonyloxy or trimethylsilyl.

“Pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid; or, for example a salt of a compound of Formula I which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. All tautomers, where possible, are included within the scope of the invention.

The present invention also encompasses prodrugs of a compound of formula I that is compounds which are converted into a compound of formula I in vivo.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl and t-butyl . Preferred alkyl groups are methyl, ethyl, propyl, isopropyl and tertiary butyl.

Unless otherwise stated or indicated, the term “alkoxy” denotes a group 0-alkyl, wherein alkyl is as defined above.

Unless otherwise stated or indicated, the term “halo” shall mean fluorine, chlorine, bromine or iodine.

Specific compounds of the invention are one or more of the following:
1-[4(benzyloxy)phenyl]-5-(2,4-dichlorophenyl)-2-methyl-N-piperidin-1-yl-1H-pyrrole-3-carboxamide;
4-{5-(2,4-dichlorophenyl)-2-methyl-3-[piperidin-1-ylamino)carbonyl]-1H-pyrrol-1-yl}phenyl trifluoromethanesulfonate;
5-(2,4-dichlorophenyl)-2-methyl-N-piperidin-1-yl-1-(4-(3,3,3-trifluoropropoxyphenyl))-1H-pyrrole-3-carboxamide;
4-{5-(2,4-dichlorophenyl)-2-methyl-3-[(piperidin-1-ylamino)carbonyl]-1H-pyrrol-1-yl}phenyl butane-1-sulfonate;
5-(2,4-Dichloro-phenyl)-2-methyl-1-(4-trimethyl silanyl-phenyl)-1H-pyrrole-3-carboxylic acid piperidin-1-ylamide; and
4-{5-(2,4-dichlorophenyl)-2-methyl-3-[(piperidin-1-ylamino)carbonyl]-1H-pyrrol-1-yl}phenyl propane-1-sulfonate
   as well as pharmaceutically acceptable salts thereof.

### Methods of Preparation

The compounds of the invention may be prepared as outlined below according to any of the following methods. However, the invention is not limited to these methods, the compounds may also be prepared as described for structurally related compounds in the prior art.

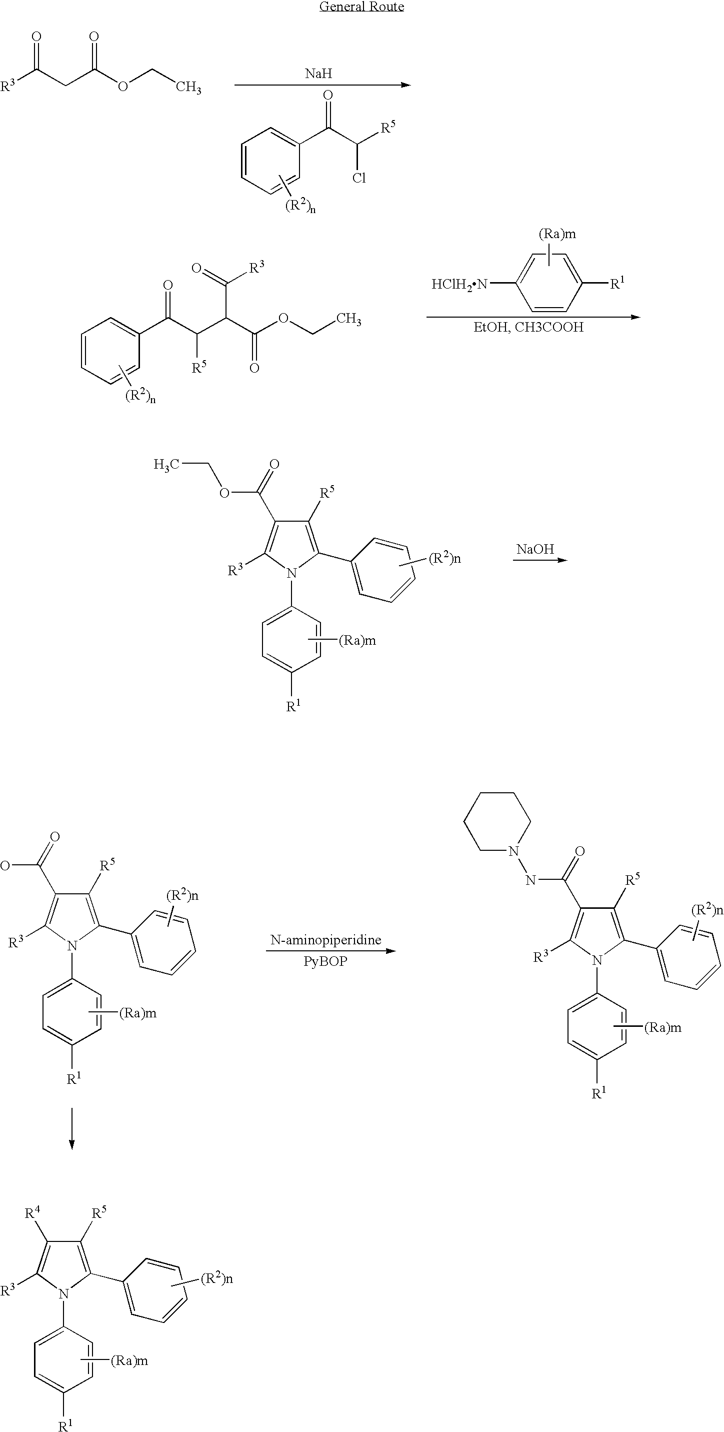

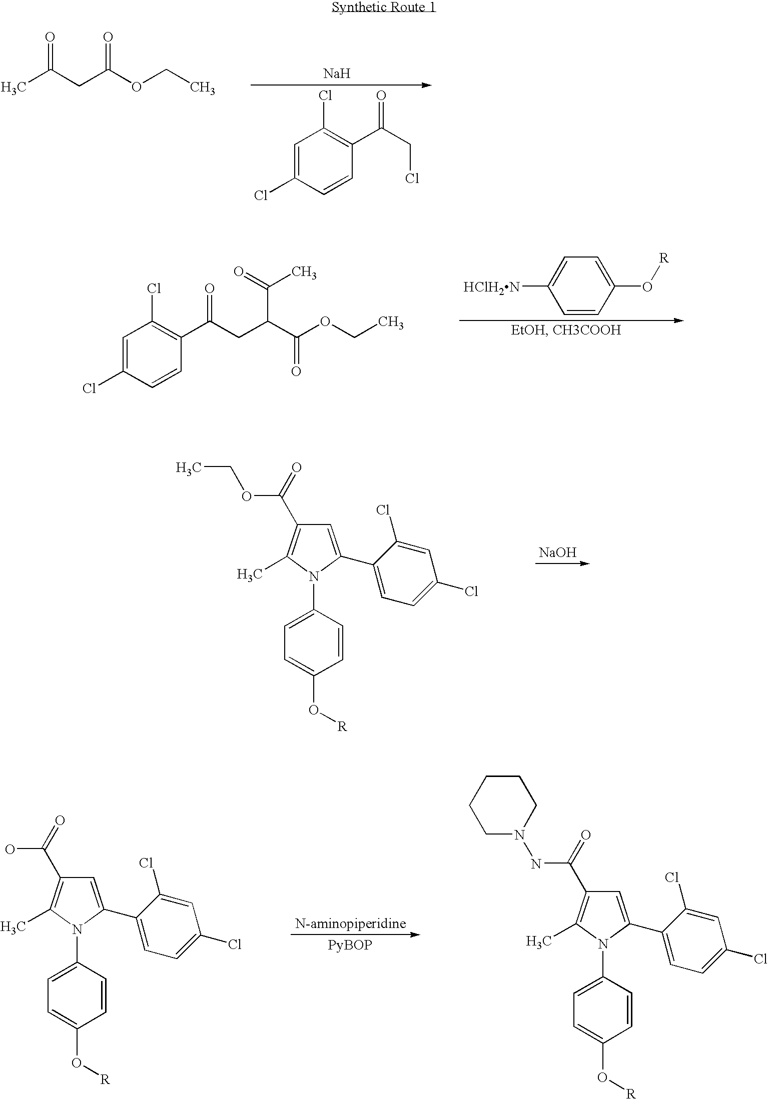

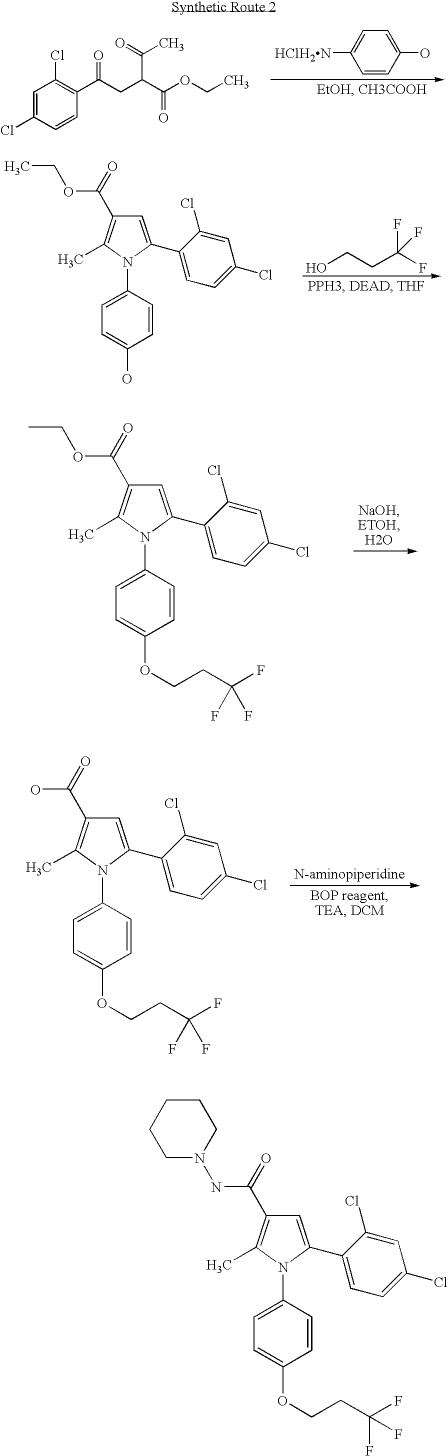

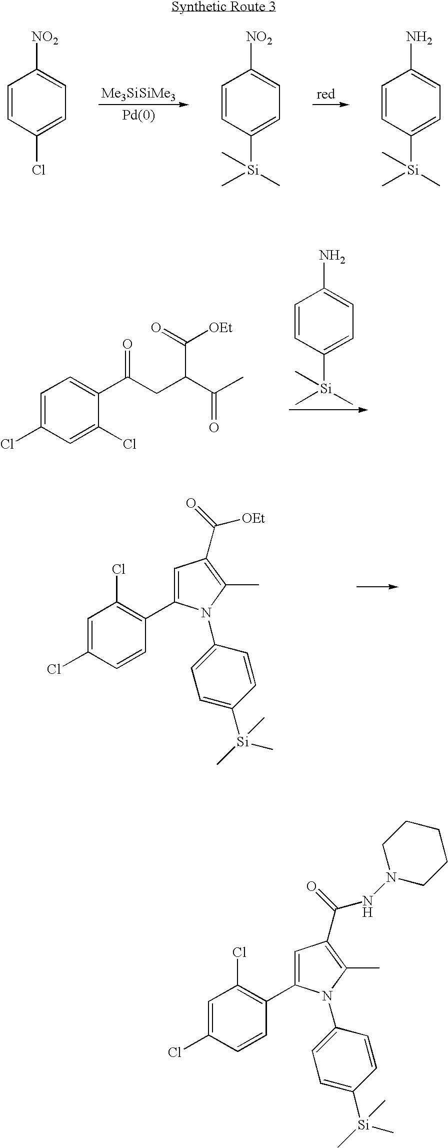

### Certain intermediate compounds are believed to be novel and form part of the present invention.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compounds are also potentially useful for the treatment of immune, cardiovascular disorders (e.g. atherosclerosis, arteriosclerosis, angina pectoris, abnormal heart rythms, and arrythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of formula I in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of formula I in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of a compound of formula I in the preparation of a medicament for the treatment or prophylaxis of immune, cardiovascular disorders (e.g. atherosclerosis, arteriosclerosis, angina pectoris, abnormal heart rythms, and arrythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfinction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of formula I in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disoerders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of formula I to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of a compound of formula I to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse. In a still further aspect the present invention provides a method comprising administering a pharmacologically effective amount of a compound of formula I to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma. In a still further aspect the present invention provides a method comprising administering a pharmacologically effective amount of a compound of formula I to a patient in need thereof for the prophylaxis or treatment of immune, cardiovascular disorders (e.g. atherosclerosis, arteriosclerosis, angina pectoris, abnormal heart rythms, and arrythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of formula I to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particulary suitable for the treatment of obesity or being overweight, (e.g., promotion of weight loss and maintenance of weight loss), prevention or reversal of Weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items). The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders(e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially usefuil as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I to a patient in need thereof.

The compounds of the present invention are particulary suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s).The compounds of the present invention may also be used to prevent or reverse weight gain associated with smoking cessation

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose. regulators and alpha-glucosidase inhibitors). In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:
a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator;
an orexin receptor modulator;
a phosphoinositide-dependent protein kinase (PDK) modulator; or
modulators of nuclear receptors for example LXR, FXR, RXR, GR, ERRα, β, PPARα, β, γ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a ghrelin/ghrelin receptor modulator;
a DPP-IV inhibitor;
   or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:
a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to a further aspect of the present invention there is provided a kit comprising:
a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the the treatment of obesity and its associated complications in a warm -blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

### Pharmacological Activity

Compounds of the present invention are active against the receptor product of the CB1 gene. The affinity of the compounds of the invention for central cannabinoid receptors is demonstrable in methods described in Devane et al , Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B-A)/1+((C/x) UD)) and the IC50 value determined as the concentration required to give half maximal inhibition of GTPγS binding under the conditions used.

The compounds of the present invention are active at the CBI receptor (IC50<1 micromolar). Most preferred compounds have IC50<200 nanomolar. For example the IC50 of Example 10 is 6.0 nM and Example 11 is 6.4 nM.

The compounds of the invention are believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example higher free fraction of drug) or solubility compared to representative reference CB1 antagonists/inverse agonist agents.

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### Examples

### Abbreviations

DCM—dichloromethane
DMF—dimethylformamide
DMAP—4-dimethylaminopyridine
EDC—1-(3-dimethylaminopropyl)-3-ethylcarbodiimide
TEA—triethylamine
TFA—trifluoroacetic acid
DMSO—dimethyl sulfoxide
DEA—Diethylamine
PCC—Pyridinium chlorochromate
PyBOP—benzotriazol-1-yl-oxytri-pyrrolidinophosphonium hexafluorophosphate
HBTU—O-Benzotriazol-1-yl-N,N,N′,N′-tetramethyluronium Hexafluorophosphate
DAST—(diethyl amino)sulphur trifluoride
DIEA—N,N-diisopropylethylamine
t triplet
s singlet
d doublet
q quartet
qvint quintet
m multiplet
br broad
bs broad singlet
dm doublet of multiplet
bt broad triplet
dd doublet of doublet

### General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC with a mass triggered fraction collector, Shimadzu QP 8000 single quadrupole mass spectrometer equipped with 19×100 mm C8 column. The mobile phase used was, if nothing else is stated, acetonitrile and buffer (0.1 M NH₄Ac:acetonitrile 95:5).

For isolation of isomers, a Kromasil CN E9344 (250×20 mm i.d.) column was used. Heptane:ethyl acetate:DEA 95:5:0.1 was used as mobile phase (1 ml/min). Fraction collection was guided using a UV-detector (330 nm).

### EXAMPLES OF THE INVENTION

### Example 1

### Step A Ethyl 2-Acetyl-4-(2,4-dichlorophenyl)-4-oxobutanoate

Ethylacetoacetate (6.0 mL, 47.4 mmol) was added to a suspension of sodium hydride (3.0 g, 60% by weight, 75 mmol) in THF (250 mL) under N₂ and after 15 minutes, 2, 2′, 4′-trichloroacetophenone (15.0 g, 67.1 mmol) was added. After stirring at room temperature for 18 h, the reaction was quenched by adding saturated aq NH₄Cl and extracted with ethyl acetate. The organic layer was dried (MgSO₄) and concentrated. The residue was purified by flash column chromatography (10:1 hexanes/EtOAc) to afford Ethyl 2-Acetyl-4-(2,4-dichlorophenyl)-4-oxobutanoate as an oil (5.6 g, 37%):¹H NMR (300 MHz, CDCl₃) δ 7.60 (d, J=8.3 Hz, 1H), 7.50 (d, J=1.9 Hz, 1H), 7.30 (dd, J=8.4, 2.0 Hz, 1H), 4.00-4.20 (m, 3H), 3.20-3.50 (m, 2H), 2.20 (s, 3H), 1.10-1.30 (m, 3H); ESI MS m/z 317 [C₁₄H₁₄Cl₂O₄+H]⁺.

### Step B Ethyl 1-[4-benzyloxy)phenyl]-5-(2,4-dichlorophenyl)-2-methyl-1H-pyrrole-3-carboxylate

A solution of Ethyl 2-Acetyl-4-(2,4-dichlorophenyl)-4-oxobutanoate from Exl, Step A (2.85 g, 9.0 mmol) and 4-benzyloxyaniline hydrochloride (2.14 g, 9.1 mmol) in 1:1 ethanol/acetic acid (80 mL) was heated at reflux for 18 H. After cooling, the solution was partially concentrated and diluted with ethyl acetate. It was washed with saturated NaHCO₃ solution, and the organic layer was dried (MgSO₄) and concentrated. The residue was purified by flash column chromatography (10:1 hexanes/EtOAc) to afford the title compound as a white solid (1.67 g, 39%): ¹H NMR (300 MHz, CDCl₃) δ 6.90-7.40 (m, 12H), 6.73 (s, 1H), 5.02 (s, 2H), 4.31 (q, J=7.1 Hz, 2H), 2.40 (s, 3H), 1.36 (t, J=7.1 Hz); ESI MS m/z 480 [C₂₇H₂₃Cl₂NO₃+H]⁺; HPLC (Method A) 99.6% (AUC), t_{R}=36.2 min.

### Step C 1-[4-benzyloxv)phenyl]-5-(2,4-dichlorophenyl)-2-methyl-1H-2pyrrole-3-carboxylic acid

Ethyl 1-[4-benzyloxy)phenyl]-5-(2,4-dichlorophenyl)-2-methyl-1H-pyrrole-3-carboxylate from EX1, Step B (400 mg, 0.833 mmol) and sodium hydroxide (1.417 g, 35.42 mmol) were refluxed in ethanol (20 ml) 1.5 hour. The solvent was evaporated and the mixture redissolved in water and neutralised with HCl (4M). The product was collected by filtration, washed with water (500 ml) and air dried over night. The crude product (375 mg, 99%) was used in steps described below without further purification.

¹H NMR (399.964 MHz) δ 7.45-7.10 (m, 6H), 7.10-6.75 (m, 7H), 5.00 (s, 2H), 4.00-3.00 (br, 1H), 2.37 (s, 3H).

¹³C NMR(100.580MHz)δ 172.87, 158.05, 136.84, 136.66, 135.31, 133.89, 133.49, 131.21, 130.95, 129.51, 129.23, 128.76, 128.45, 128.28, 127.86, 126.48, 116.77, 114.84, 112.91, 70.32, 12.74.

MS m/z 452, 454, 456 (M+H)⁺.

### Step D 1-[4(benzyloxy)phenyl]-5 -(2,4-dichlorolphenyl)-2-methyl-N-piperidin-1-yl-1H-pyrrole-3-carboxamide

1-[4-benzyloxy)phenyl]-5-(2,4-dichlorophenyl)-2-methyl-1H-pyrrole-3-carboxylic acid from Ex1,Step C (174 mg, 0.385 mmol) was dissolved in DCM (1 ml) and TEA (0.5 ml) under N₂ (g) and cooled to −78° C. Benzotriazol-1-yl-oxytri-pyrrolidinophosphonium hexafluorophosphate dissolved in DCM (0.5 ml) was added dropwise followed immedately by the addition of 1-aminopiperidine (45 mg, 0.454 mmol). The reaction was continued at −78° C. under N₂ (g) for 1 hour and then at room temperature over night. The mixture was extracted with water and dried over MgSO₄. Finally the product was purified by flash chromatography (SiO₂, toluene:ethylacetate 9:1) to give a slightly yellow powder (98 mg, 48%).

¹H NMR (399.964 MHz) δ 7.45-7.10 (m, 6H), 7.10-6.80 (m, 6H), 6.65-6.55 (br, 1 H), 6.45-6.35 (br, 1H), 5.00 (s, 2H), 3.00-2.80 (br, 4H), 2.40 (s, 3H), 1.80-1.65 (br, 4H), 1.50-1.35 (br, 2H).

¹³C NMR (100.580 MHz) δ 163.69, 158.49, 136.59, 136.19, 135.45, 134.32, 133.75, 130.69, 130.45, 129.57, 129.42, 128.83, 128.44, 128.39, 127.80, 126.73, 115.20, 114.25, 108.37, 70.47, 57.48, 25.76, 23.58, 12.61. MS m/z 534, 536, 538 (M+H)⁺.

### Example 2

### Step A 5-(2,4-dichlorophenyl)-1-(4-hydroxyphenyl)-2-methyl-N-piperidin-1-yl-1H-pyrrole-3-carboxamide

1-[4(benzyloxy)phenyl]-5-(2,4-dichlorophenyl)-2-methyl-N-piperidin-1-yl-1H-pyrrole-3-carboxamide from Example 1, Step D (98 mg, 0.1 83 mmol) and dimethyl sulfide (70 mg, 1.126 mmol) were dissolved in DCM (3 ml). Boron trifluoride etherate (224 mg, 1.578 mmol) was added dropwise and the reaction continued at room temperature 24 hours. The mixture was extracted with water and dried over MgSO₄. The crude product (77 mg, 95%) was used in steps described below without further purification.

¹H NMR (399.964 MHz) δ 7.32-7.24 (m, 1H), 7.10-6.95 (m, 2H), 6.95-6.85 (m, 2H), 6.80-6.75 (m, 2H), 6.68 (s, 1H), 6.43 (s, 1H), 3.35-3.25 and 3.07-2.97 (two multiplets, 3H), 2.90-2.77 (br, 1H), 2.40 (s, 3H), 2.10-1.82 and 1.75-1.50 (two multiplets, 6H), 1.40-1.30 (m, 1 H).

¹³C NMR (100.580 MHz) δ 167.07, 156.10, 136.07, 135.59, 134.40, 133.77, 130.62, 130.22, 129.88, 129.48, 126.67, 115.99, 110.69, 108.48, 57.40, 25.34, 22.50, 12.74. MS m/z 444, 446, 448 (M+H)⁺.

### Step B 4-{5-(2,4-dichlorophenyl)-2-methyl-3-[piperidin-1-ylamino)carbonvl]-1H-pyrrol-1-yl}phenyl trifluoromethanesulfonate

5-(2,4-dichlorophenyl)-1-(4-hydroxyphenyl)-2-methyl-N-piperidin-1-yl-1H-pyrrole-3-carboxamide, Ex2, Step A (44 mg, 0.099 mmol) was dissolved in DCM (3 ml) and TEA (40 μl) and cooled to −78° C. Trifluoromethane sulfuric anhydride (350 μl, 0.208 mmol) was added and the reaction continued at −78° C. for 1 hour. The mixture was extracted with cold NaHCO₃ (aq) and water and dried over MgSO₄. The product was purified by flash chromatography (SiO₂, toluene:ethylacetate 9:1) and preparatory HPLC (kromasil C8 column, ammonium acetate (aq, 0.1 M):acetonitrile, product came at about 80% acetonitrile) to give the subtitle compound as a slightly yellow powder (3 mg, 6%).

¹H NMR (399.964 MHz) δ 7.35-6.95 (m, 7H), 6.70-6.40 (br, 1H), 6.41 (s, 1H), 2.88 (s, 4H), 2.43 (s, 3H), 1.85-1.70 (s, 4H), 1.50-1.40 (s, 2H). MS m/z 576, 578, 580 (M+H)⁺.

### Example 3

### Step A. Ethyl 5-(2,4-dichlorophenyl)-1-(4-hydroxyphenyl)-2-methyl-1H-pyrrole-3-carboxylate

A solution of Ethyl 2-Acetyl-4-(2,4-dichlorophenyl)-4-oxobutanoate, from Ex1, Step A (5.45 g, 17.18 mmol), 4-aminophenol (2.40 g, 21.99 mmol), and acetic acid (10 mL) in ethanol (15 mL) was heated at reflux for 14 hours. After cooling, the reaction was quenched by adding saturated NaHCO₃ solution and extracted into EtOAc. The organic layer was dried (MgSO₄), and concentrated. The residue was purified by flash column chromatography (silica gel, 9:1 hexanes/EtOAc) to afford Ethyl 5-(2,4-dichlorophenyl)-1-(4-hydroxyphenyl)-2-methyll-1H-pyrrole-3-carboxylate (2.87 g, 43%) as an oil. A portion of this material was recrystallized from hexanes/ethyl acetate to afford Ethyl 5-(2,4-dichlorophenyl)-1-(4-hydroxyphenyl)-2-methyll-1H-pyrrole-3-carboxylate as a white solid.: ¹H NMR (300 MHz, CDCl₃) δ 7.25-7.30 (m, 1H), 7.00-7.05 (m, 2H), 6.80-6.95 (m, 2H), 6.70-6.75 (m, 3H), 6.30-6.40 (broad s, 1H), 4.32 (q, J=6.9 Hz, 2H), 2.38 (s, 3H), 1.36 (t, J=7.2 Hz, 3H); ESI MS m/z 394 [C₂₀H₁₇Cl₂NO₃+H]⁺.

### Step B. Ethyl 5-(2,4-dichlorophenyl)-2-methyl-1-(4-(3,3,3 -trifluoropropoxvlhenyl))-1H-pyrrole-3-carboxylate

A solution of Ethyl 5-(2,4-dichlorophenyl)-1-(4-hydroxyphenyl)-2-methyll-1H-pyrrole-3-carboxylate, from Ex3, Step A (2.87 g, 7.35 mmol) in THF (30 mL) was treated with 3,3,3-trifluoropropan-1-ol (0.65 mL, 7.37 mmol), triphenylphosphine (1.94 g, 7.40 mmol) and diethylazodicarboxylate (1.20 mL, 7.72 mmol) at 0° C. The resulting solution was stirred at room temperature for 14 hours. The solution was concentrated under reduced pressure and the residue was taken in ethyl acetate. This solution was washed with water and the organic layer was dried (MgSO₄), and concentrated to afford the crude product. The crude product was purified by flash column chromatography (silica gel, 8:1 hexanes/EtOAc) to afford Ethyl 5-(2,4-dichlorophenyl)-2-methyl-1-(4-(3,3,3-trifluoropropoxyphenyl))-1H-pyrrole-3-carboxylate (0.90 g, 25%): ¹H NMR (300 MHz, CDCl₃) δ 7.29 (d, J=1.9 Hz, 1H), 7.00-7.10 (m, 4H), 6.80 (d, J=8.9 Hz, 2H), 6.71 (d, J=1.5 Hz, 1H), 4.30 (q, J=7.1 Hz, 2H), 4.10-4.20 (m, 2H), 2.55-2.65 (m, 2H), 2.38 (s, 3H), 1.36 (t, J=7.1 Hz, 3H); ESI MS m/z 486 [C₂₄H₂₂Cl₂F₃NO₂+H]⁺.

### Step C. 5-(2,4-dichlorophenyl)-2-methyl-1-(4-(3,3,3-trifluoropropoxyphenyl))-1H-pyrrole-3-carboxylic acid

A solution of Ethyl 5-(2,4-dichlorophenyl)-2-methyl-1-(4-(3,3,3-trifluoropropoxyphenyl))-1H-pyrrole-3-carboxylate, from Ex3, StepB (0.96 g, 1.97 mmol) in ethanol (20 mL) was combined with a 1.0 M solution of NaOH (10 mL, 10.0 mmol). The resulting solution was heated at reflux for 16 hours. It was then poured into ice-cold 1 N HCI solution and extracted into EtOAc. The organic layer was dried (MgSO₄) and concentrated to afford 5-(2,4-dichlorophenyl)-2-methyl-1-(4-(3,3,3-trifluoropropoxyphenyl))-1 H-pyrrole-3-carboxylic acid (0.75 g, 83%) as a yellow powder: ¹H NMR (300 MHz, CD₃OD) δ 7.36 (s, 1H), 7.15-7.17 (m, 3H), 7.05-7.10 (m, 2H), 6.90-7.00 (m, 3H), 6.71 (d,J=8.7 Hz, 1H), 6.62 (d, J=3.9 Hz, 1H), 4.19 (t, J=6.0 Hz, 2H), 2.65-2.70 (m, 2H), 2.35 (s, 3H), 1.90-1.95 (m, 2H); ESI MS m/z 458 [C₂₅H₂₆ClN₃O₄+H]⁺.

### Step D. 5-(2,4-dichlorophenyl)-2-methyl-N-piperidin-1-yl-1-(4-(3,3,3-trifluoropropoxyphenyl))-1H-pyrrole-3-carboxamide

A solution of 5-(2,4-dichlorophenyl)-2-methyl-1-(4-(3,3,3-trifluoropropoxyphenyl))-1H-pyrrole-3-carboxylic acid, from Ex3, StepC (0.64 g, 1.40 mmol) in CH₂Cl₂ (5 mL) under N₂ was treated with 1-aminopiperidine (0.19 mL, 1.76 mmol), BOP reagent (1.04 g, 2.35 mmol) and triethylamine (0.65 mL, 4.66 mmol). The solution was stirred at room temperature for 2 days. It was washed with water and the organic layer was dried (MgSO₄), and concentrated to afford the crude product. The crude product was purified by flash column chromatography (silica gel, 2:3 hexanes/EtOAc) to afford 5-(2,4-dichlorophenyl)-2-methyl-N-piperidin-1-yl-1-(4-(3,3,3 -trifluoroproloxyphenyl))-1H-pyrrole-3-carboxamide (0.22 g, 30%) as a white powder: M.P. 237-239° C: ¹H NMR (300 MHz, CDCl₃) 67 7.37 (s, 1H), 7.18 (d, J=0.5 Hz, 1H), 7.07 (d, J=6.7 Hz, 2H), 7.05 (d, J=3.2 Hz, 2H), 6.60 (s, 1H), 4.19 (t, J=6.2 Hz, 2H), 2.80-2.85 (m, 4H), 2.60-2.70 (m, 2H), 2.33 (s, 3H), 1.70-1.75 (m, 4H), 1.40-1.45 (m, 2H); ESI MS m/z 540 [C₂₆H₂₆Cl₂F₃N₃O₂+H]⁺; HPLC (Method A) 89.3% (AUC), t_{R}=17.8 minutes.

### Example 4

4-{5-(2,4-dichlorophenyl)-2-methyl-3-[(piperidin-1-ylamino)carbonyl]-1H-pyrrol-1-yl}phenyl butane-1-sulfonate

5-(2,4-dichlorophenyl)-1-(4-hydroxyphenyl)-2-methyl-N-piperidin-1-yl-1H-pyrrole-3-carboxamide, from Ex 2, Step A (66 mg, 0.147 mmol) from (b) and DMAP (27 mg, 0.221 mmol) were dissolved in dry DCM (2 ml) under N₂ (g). TEA (100 μl, 0.717 mmol) and 1-Butansulfonyl chloride (40 μl, 0.311 mmol) were added and the reaction continued at room temperature for 6 hours under N₂ (g). The mixture was extracted with water and dried over MgSO₄. The product was purified by preparatory HPLC (kromasil C8 column, ammonium acetate (aq, 0.1 M):acetonitrile, product came at about 100% acetonitrile) to give the subtitle compound as a white powder (42 mg, 50%).

¹H NMR (399.964 MHz) δ 7.35-6.95 (m, 7H), 6.70-6.40 (br, 1H), 6.40 (s, 1H), 3.24 (t, 2H), 2.98-2.78 (br, 4H), 2.42 (s, 3H), 2.02-1.88 (m, 2H), 1.82-1.68 (br, 4H), 1.57-1.43 (m, 2H), 1.50-1.38 (br, 2H), 0.97 (t, 3H).

¹³C NMR(100.580MHz)δ 163.41, 148.48, 136.19, 135.85, 135.45, 134.81, 133.69, 130.23, 129.84, 129.70, 129.16, 126.93, 122.68, 114.94, 108.96, 57.48, 50.88, 25.75, 25.63, 23.57, 21.59, 13.64, 12.65. MS m/z 564, 566, 568 (M+H)⁺.

### Example 5

### Step A. 4-(Trimethylsilyl)-1-nitrobenzene

1-Chloro-4-nitrobenzene (2.25 g, 14.3 mmol), hexamethyldisilane (8.98 g, 61.3 mmol) and tetrakis(triphenylphosphine)palladium(0) (450 mg, 0.39 mmol) in xylene (7 ml) was sealed under nitrogen and stirred at 160° C. for 4 hours. The mixture was cooled, 100 ml hexane was added, and the mixture filtered through a pad of Celite. Evaporation of the filtrate gave a dark oil. Flash-chromatography (silica, hexane:CH₂Cl₂ 95:5, 90:10) afforded 1.63 g (68%) of the title compound.

¹H NMR (CDCl₃): δ 8.19 (2H, d), 7.69 (2H, d), 0.34 (9H, s)

### Step B. 4-(Trimethylsilyl)aniline

4-(Trimethylsilyl)-1-nitrobenzene, Ex 5, Step A (1.63 g, 8.35 mmol) dissolved in ethanol (50 ml) was added 5% palladium on charcoal (500 mg, 0.23 mmol), and stirred under 1 atm of hydrogen pressure overnight. The mixture was then filtered through a pad of Celite and concentrated under reduced pressure, giving 1.3 g (94%) of the title compound.

### Step C. 5-(2,4-Dichloro-phenyl)-2-methyl-1-(4-trimethylsilanyl-phenyl)-1H-pyrrole-3-carboxylic acid ethyl ester

2-[2-(2,4-Dichloro-phenyl)-2-oxo-ethyl]-3-oxo-butyric acid ethyl ester, from Ex1, Step A (1.36 g, 4.3 mmol) and 4-(Trimethylsilyl)aniline (0.71 g, 4.3 mmol) was stirred at 110° C. for 72 hours. Flash-chromatography (silica, hexane:EtOAc 95:5, 90:10) afforded 217 mg (12%) of the title compound.

¹H NMR (CDCl₃): δ 7.46 (2H, d), 7.31 (2H, m), 7.12-7.04 (4H, m), 4.34 (2H, q), 2.43 (3H, s), 1.39 (3H, t), 0.28 (9H, s) MS m/z 469 (M+Na)

### Step D. 5 -(2,4-Dichloro-phenyl)-2-methyl-1-(4-trimethylsilanyl-phenyl)-1H-pyrrole-3-carboxylic acid piperidin-1-ylamide

To a solution of aminopiperidine (133 μl, 1.23 mmol) in dry chloroform (2 ml), was added dropwise under nitrogen a solution of trimethylaluminum in toluene (613 μl, 2 M sol., 1.23 mmol). The mixture was kept at r.t. with stirring for an additional 1 h. 5-(2,4-Dichloro-phenyl)-2-methyl-1-(4-trimethylsilanyl-phenyl)-1H-pyrrole-3-carboxylic acid ethyl ester (217 mg, 0.49 mmol) dissolved in dry chloroform (1 ml) was then added and the solution was warmed to 45° C. and stirred for 19 hours under nitrogen. The reaction mixture was poured carefully into 10 ml of 2 M HCl and the resultant mixture was stirred at 40 ° C. for 30 min. The layers were separated and the aqueous layer was extracted with chloroform (2×15 ml). The combined organic layers were washed with brine, dried (Na₂SO₄) and concentrated under reduced pressure. Flash-chromatography (silica, hexane:EtOAc 80:20) afforded 36 mg (16%) of the title compound as a white solid.

¹H NMR (CDCl₃): δ 7.47 (2H, d), 7.33 (2H, d), 7.08-7.00 (4H, m), 2.92 (4H, m), 2.45 (3H, s), 1.77 (4H, m), 1.48 (2H, m), 0.29 (9H, s) MS m/z 523 (M+Na) HPLC: 92.4%.

1. A compound of formula (I) 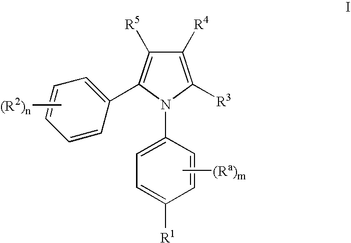
or a pharmaceutically acceptable salt thereof, in which
R¹ represents
a) a C₃₋₆alkoxy group substituted by one or more fluoro,
b) a group of formula phenyl(CH₂)ₚO— in which p is 1, 2 or 3 and the phenyl ring is optionally substituted by 1, 2 or 3 groups represented by Z,
c) a group R⁶S(O)₂O or R⁶S(O)₂NH in which R⁶ represents a C₁₋₆alkyl group optionally substituted by one or more fluoro, or R⁶ represents phenyl or a heteroaryl group each of which is optionally substituted by 1, 2 or 3 groups represented by Z, or
d) a group of formula (R⁷)₃Si in which R⁷ represents a C₁₋₆alkyl group which may be the same or different;
R^{a} represents halo, a C₁₋₃alkyl group or a C₁₋₃alkoxy group, and m is 0, 1, 2 or 3;
R² represents a C₁₋₃alkyl group, a C₁₋₃alkoxy group, hydroxy, nitro, cyano or halo, and n is 0, 1, 2 or 3;
R³ represents H, a C₁₋₆alkyl group, a C₁₋₆alkoxy group or a C₁₋₆alkoxyC₁₋₆alkylene group which contains a maximum of 6 carbon atoms, each of which groups is optionally substituted by one or more fluoro or cyano;
R⁴ represents
a) a group X—Y-NR⁸R⁹ in which
X is CO or SO₂;
Y is absent or represents NH optionally substituted by a C₁₋₃alkyl group; and
R⁸ and R⁹ independently represent:
a C₁₋₆alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂),(phenyl ), in which r is 0,1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)tHet in which t is 0,1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo;
or R⁸ represents H and R⁹ is as defined above;
or R⁸ and R⁹ together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro or benzyl; or
b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁵ represents H or a C₁₋₃alkyl group;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperdinyl or piperazinyl in which the heterocyclic amine is optionally substituted by a C₁₋₃alkyl group or hydroxyl.

2. A compound of formula (IA) 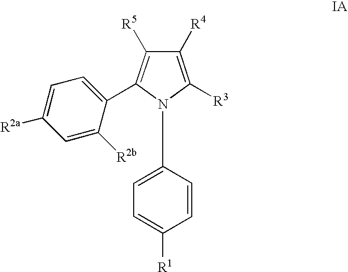
in which
R¹ is
a) a C₃₋₆alkoxy group substituted by one or more fluoro,
b) a group of formula phenyl(CH₂)ₚO— in which p is 1, 2 or 3 and the phenyl ring is optionally substituted by 1, 2 or 3 groups represented by Z,
c) a group R⁶S(O)₂O or R⁶S(O)₂NH in which R⁶ represents a C₁₋₆alkyl group optionally substituted by one or more fluoro, or R⁶ represents phenyl or a heteroaryl group each of which is optionally substituted by 1, 2 or 3 groups represented by Z, or
d) a group of formula (R⁷)₃ Si in which R⁷ represents a C₁₋₆alkyl group which may be the same or different;
R^{2a} represents chloro;
R^{2b} represents chloro;
R³ represents a C₁₋₃alkyl group;
R⁴ represents a group CONHNR⁸R⁹ in which NR⁸R⁹ represents piperidino; and
R⁵ represents H.

3. A compound selected from
[4(benzyloxy)phenyl]-5-(2,4-dichlorophenyl)-2-methyl-N-piperidin-1-yl-1H-pyrrole-3-carboxamide;
4-{5-(2,4-dichlorophenyl)-2-methyl-3-[piperidin-1-ylamino)carbonyl]-1H-pyrrol-1-yl}phenyl trifluoromethanesulfonate;
5-(2,4-dichlorophenyl)-2-methyl-N-piperidin-1-yl-1-(4-(3,3,3-trifluoropropoxyphenyl))-1H-pyrrole-3-carboxamide;
4-{5-(2,4-dichlorophenyl)-2-methyl-3-[(piperidin-1-ylamino)carbonyl]-1H-pyrrol-1-yl}phenyl butane-1-sulfonate;
5-(2,4-Dichloro-phenyl)-2-methyl-1-(4-trimethylsilanyl-phenyl)-1H-pyrrole-3-carboxylic acid piperidin-1-ylamide; and
4-{5-(2,4-dichlorophenyl)-2-methyl-3-[(piperidin-1-ylamino)carbonyl]-1H-pyrrol-1-yl}phenyl propane-1-sulfonate;
or a pharmaceutically acceptable salts thereof.

4. (canceled)

5. A pharmaceutical composition comprising a compound of formula I or formula IA according to any one of claims 1 to 3 and a pharmaceutically acceptable adjuvant, diluent or carrier.

6. (canceled)

7. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications in a warm blooded animal in need thereof, comprising administering to said animal a pharmacologically effective amount of a compound of formula I or formula IA according to any one of claims 1 to 3.

8. (canceled)

9. A method of treating obesity in a warm blooded animal in need thereof, comprising administering to said animal a pharmacologically effective amount of a compound of formula I or formula IA according to any one of claims 1 to 3. _
